(19) United States
(12) Patent Application Publication
(10) Pub. No.: US 2019/0254696 A1
(43) Pub. Date: Aug. 22, 2019

(54) TOOL BIT FOR AN ULTRASONIC OSTEOTOME

(71) Applicant: JIANGSU SMTP TECHNOLOGY CO., LTD., Jiangsu (CN)

(72) Inventors: Xianze SUN, Jiangsu (CN); Songtao ZHAN, Jiangsu (CN); Zhen FENG, Jiangsu (CN); Qun CAO, Jiangsu (CN)

(21) Appl. No.: 16/286,709

(22) Filed: Feb. 27, 2019

Related U.S. Application Data
(63) Continuation of application No. PCT/CN2017/097689, filed on Aug. 16, 2017.

(30) Foreign Application Priority Data
Aug. 31, 2016 (CN) 201621022534.5

(51) Int. Cl. A61B 17/32; A61B 17/16

(52) U.S. Cl. CPC A61B 17/320068; A61B 2017/320075; A61B 17/1659; A61B 17/1615

(57) ABSTRACT

Disclosed is a tool bit for an ultrasonic osteotome comprising a head portion of the tool bit, an arbor and a bit body, with one end of the arbor being connected with the head portion of the tool bit, and the other end of the arbor being connected with the bit body wherein the head portion of the tool bit has a flat-sheet shape, and the head portion of the tool bit is bent about an axis in an axial direction of the tool bit, with an inner surface and an outer surface of the flat-sheet head portion of the tool bit having a same radius of curvature, and the head portion of the tool bit is provided with a curved part. When the tool bit for an ultrasonic osteotome is used for osteotomy, it can conveniently and quickly cut a hole in the vertebral plate or other bone tissue.

### CROSS REFERENCE TO RELATED APPLICATIONS

This is a Continuation application of International Application Ser. No. PCT/CN2017/097689, filed Aug. 16, 2017, which claims the benefit of Chinese Application No. 201621022534.5, filed Aug. 31, 2016, the disclosures of which are hereby incorporated by reference.

### BACKGROUND

The present disclosure relates to the field of medical instruments and devices, and in particular to an ultrasonic scalpel, and more particularly to a tool bit for an ultrasonic osteotome.

In modern society, with the development of medical technology, orthopedic surgery shows a trend of diversity. Accordingly, in orthopedic surgery, ultrasonic osteotomes are often used to perform cutting, grinding, planing, scraping or arbitrary shaping on bones.

In view of the special construction of bone structure, along with the continuously development of ultrasound technology in recent years, ultrasonic osteotomes have gradually become main tools for modern orthopedic surgery. In the orthopedic surgery, a commonly used ultrasonic osteotome has a sheet-shaped tool bit, which is mainly used for cutting, as shown in FIG. 1. However, at present, these tool bits mostly have a straight sheet shape. When it is necessary to cut a hole in a bone tissue, the straight sheet tool bit has poor operability, and there is a risk of breaking the tool bit when it is used improperly, which can cause damage to other non-surgical parts and thus cause danger. This puts higher requirements on the operation level of the medical staff, places physical and mental pressure on the doctors, reduces the success rate of the operation, and increases the risk of surgery for the patient.

### BRIEF SUMMARY

The present disclosure is directed to the above problems in the prior art and provides a tool bit for an ultrasonic osteotome comprising a head portion of the tool bit, an arbor and a bit body, with one end of the arbor being connected with the head portion of the tool bit, and the other end of the arbor being connected with the bit body, wherein the head portion of the tool bit has a flat-sheet shape, and the head portion of the tool bit is bent about an axis in an axial direction of the tool bit, with an inner surface and an outer surface thereof having a same radius of curvature, and the head portion of the tool bit is provided with a curved part.

According to the tool bit for an ultrasonic osteotome of the present disclosure, preferably, the curved part is provided on a single side of the head portion of the tool bit.

According to the tool bit for an ultrasonic osteotome of the present disclosure, it is preferable that the curved part is provided with cutter teeth.

According to the tool bit for an ultrasonic osteotome of the present disclosure, preferably, the curved part is provided at the top of the head portion of the tool bit.

According to the tool bit for an ultrasonic osteotome of the present disclosure, it is preferable that the head portion of the tool bit is provided with cutter teeth on a single side thereof.

According to the tool bit for an ultrasonic osteotome of the present disclosure, preferably, the tool bit for an ultrasonic osteotome is of a hollow structure.

According to the tool bit for an ultrasonic osteotome of the present disclosure, it is preferable that a guide hole is provided at a connection portion between the arbor and the head portion of the tool bit.

According to the tool bit for an ultrasonic osteotome of the present disclosure, it is preferable that a guide slot is provided in the head portion of the tool bit to communicate with the guide hole.

According to the ultrasonic bone cutter bit of the present disclosure, preferably, the arbor and the bit body are transitioned by a beveled or curved surface.

According to the tool bit for an ultrasonic osteotome of the present disclosure, preferably, the bit body is provided with a connection mechanism at the other end thereof for connection with an ultrasonic transducer, and the bit body is provided with clamping faces on the outer surface thereof for clamping.

### BRIEF DESCRIPTION OF THE SEVERAL VIEWS OF THE DRAWINGS

In order to more clearly illustrate the technical solutions in specific embodiments of the present invention or in the prior art, the drawings used in the description of the specific embodiments or the prior art will be briefly described below. Apparently, the drawings described in the following description are only some embodiments of the present invention, and those skilled in the art can obtain other drawings based on these drawings without creative work.

FIG. 1 is a schematic structural view of a straight sheet-shaped tool bit for an ultrasonic osteotome in the prior art;

FIG. 2A is a perspective view of a tool bit for an ultrasonic osteotome according to a first embodiment of the present invention;

FIG. 2B is a front elevational view of the tool bit for an ultrasonic osteotome according to the first embodiment of the present invention;

FIG. 2C is a perspective view of the tool bit for an ultrasonic osteotome according to the first embodiment of the present invention viewed from another angle;

FIG. 3A is a perspective view of a tool bit for an ultrasonic osteotome according to a second embodiment of the present invention;

FIG. 3B is a front elevational view of the tool bit for an ultrasonic osteotome according to the second embodiment of the present invention;

FIG. 3C is a perspective view of the tool bit for an ultrasonic osteotome according to the second embodiment of the present invention viewed from another angle;

FIG. 4A is a perspective view showing a tool bit for an ultrasonic osteotome having a hollow structure according to an embodiment of the present invention;

FIG. 4B is a front elevational view showing the tool bit for an ultrasonic osteotome having the hollow structure according to the embodiment of the present invention;

FIG. 4C is a perspective view showing the tool bit for an ultrasonic osteotome having the hollow structure according to the embodiment of the present invention viewed from another angle;

FIG. 4D is a bottom plan view showing the tool bit for an ultrasonic osteotome having the hollow structure according to the embodiment of the present invention;

FIG. 4E is a front elevational view showing an internal hollow structure of the tool bit for an ultrasonic osteotome having the hollow structure according to the embodiment of the present invention;

FIG. 4F is a left side view of the tool bit for an ultrasonic osteotome having the hollow structure according to the embodiment of the present invention;

FIG. 5A is a schematic perspective view showing a tool bit for an ultrasonic osteotome having a hollow structure according to another embodiment of the present invention;

FIG. 5B is a front elevational view showing the tool bit for an ultrasonic osteotome having the hollow structure according to another embodiment of the present invention;

FIG. 5C is a schematic perspective view showing the tool bit for an ultrasonic osteotome having the hollow structure according to another embodiment of the present invention viewed from another angle;

FIG. 5D a front elevational view showing an internal hollow structure of the tool bit for an ultrasonic osteotome having the hollow structure according to another embodiment of the present invention; and

FIG. 5E is a left side view of the tool bit for an ultrasonic osteotome having the hollow structure according to another embodiment of the present invention.

### REFERENCE NUMERALS

1˜head portion of a tool bit; 2˜arbor; 3˜bit body; 4˜curved part; 5˜cutter teeth;

6˜guide hole; 7˜guide slot; 8˜clamping face.

### DETAILED DESCRIPTION

Exemplary embodiments of the present invention will be described hereinafter clearly and completely with reference to the attached drawings. Apparently, the embodiments described herein are only portions of embodiments of the invention, rather than all embodiments of the invention. It is intended that all other embodiments obtained by those skilled in the art according to the disclosed embodiments without inventive labor are all within the scope of the present invention.

In the description of the present invention, it is to be noted that the terms of “center”, “upper”, “lower”, “left”, “right”, “vertical”, “horizontal”, “internal”, “external” and the like simply indicate orientational or positional relationship based on the accompanying drawings and are used only for the purpose of facilitating and simplifying the description of the invention, rather than specifying or implying that any devices or elements indicated must have a certain orientation, be configured or operate in a certain orientation. Therefore, these terms will not be interpreted as limiting the present invention. Further, the terms of “first”, “second” and “third” and the like are only used for describing purpose, rather than being interpreted as specifying or implying relative importance.

In the description of the present disclosure, it is to be noted that, unless otherwise specified or defined clearly, the term of “attach”, “connect to”, “connect with”, “couple” and the like should be interpreted broadly. For example, they may refer to fixed connection, or detachable connection, or integral connection; they may refer to mechanical connection, or electrical connection; they may refer to direct connection, or indirect connection through an intermediate agent, or internal communication between two components. For those skilled in the art, the specific meaning of these terms in the present disclosure may be understood in combination with specific situations or contexts.

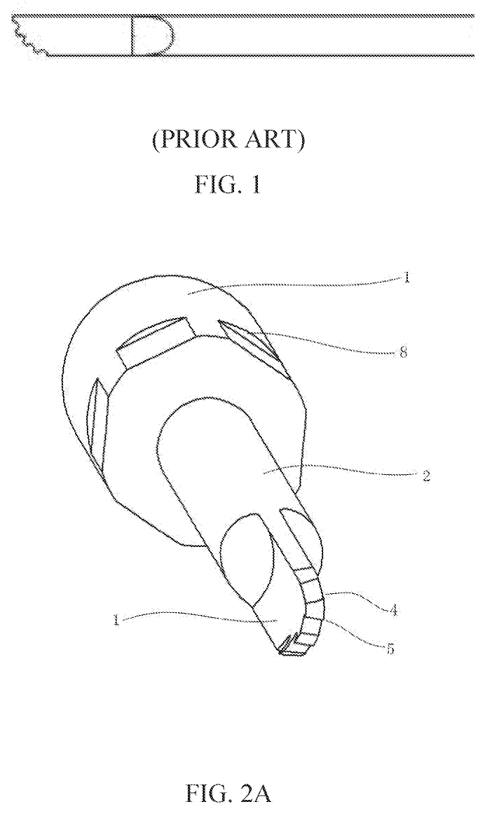

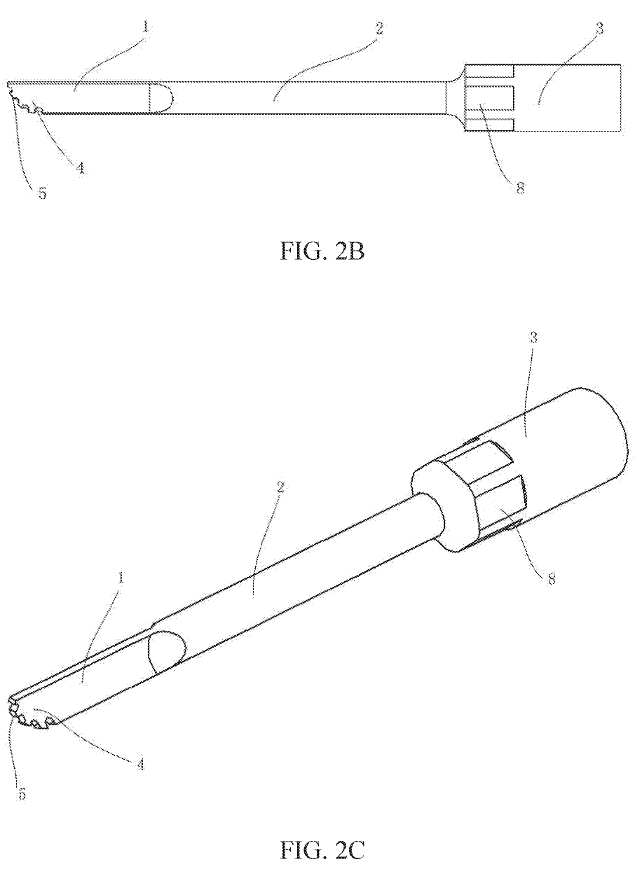

The present invention will be further described in detail below by way of specific embodiments in combination with accompanying drawings. FIG. 2A is a schematic perspective view of a tool bit for an ultrasonic osteotome according to a first embodiment of the present invention. FIG. 2B is a front view of the tool bit for an ultrasonic osteotome according to the first embodiment of the present invention. FIG. 2C is schematic perspective view of the tool bit for an ultrasonic osteotome according to the first embodiment of the present invention when viewed from another angle. As shown in FIGS. 2A to 2C, the tool bit for an ultrasonic osteotome according to the first embodiment of the present invention comprises a head portion 1 of the tool bit, an arbor 2 and a bit body 3. One end of the arbor 2 is connected with the head portion 1 of the tool bit, and the other end of the arbor 2 is connected with the bit body 3. The head portion 1 of the tool bit has a flat-sheet shape or a slightly tapered flat-sheet shape. The head portion 1 of the tool bit is entirely bent inward to a certain angle about an axis in an axial direction of the tool bit, so that the flat-sheet head portion 1 of the tool bit has an inner surface and an arcuate outer surface with a same radius of curvature. That is, the entire sheet-shaped portion of the head portion 1 of the tool bit is curved in a same arcuate degree and has a same thickness. The arcuate shape of the entire head portion 1 of the tool bit constitutes parts of concentric circles. Upon cutting, a diameter of a circle to which the flat-sheet head portion 1 belongs determines a size of a window during operation. The head portion 1 of the tool bit is provided with a curved part 4 on one end thereof. The curved part 4 is formed with cutter teeth 5 thereon. For example, a plurality of arcuate grooves may be provided to form a toothed shape. In the tool bit for an ultrasonic osteotome of the embodiment, as shown in FIGS. 2A to 2C, the bit body 3 has a cylindrical shape, and the arbor 2 has a cylindrical shape. An outer diameter of the arbor 2 is smaller than an outer diameter of the bit body 3, and there is a smooth transition between the arbor 2 and the bit body 3 by a beveled or curved surface. The smooth transition structure can effectively prevent stress concentration while avoiding hurt to the patient and the operator from sharp edges and corners. Further, the bit body 3 may be provided with clamping faces 8 for clamping, so that the operator can conveniently clamp the clamping faces 8 by using a clamping tool to screw the tool bit for an ultrasonic osteotome of the present disclosure on a transducer tightly. Preferably, the clamping faces 8 form clamping positions for a hex wrench.

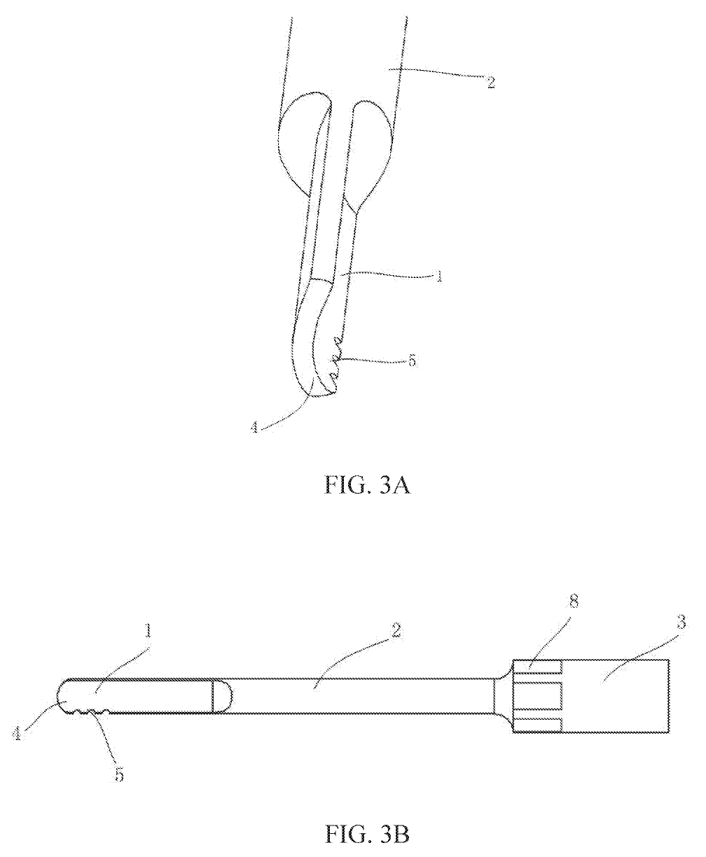

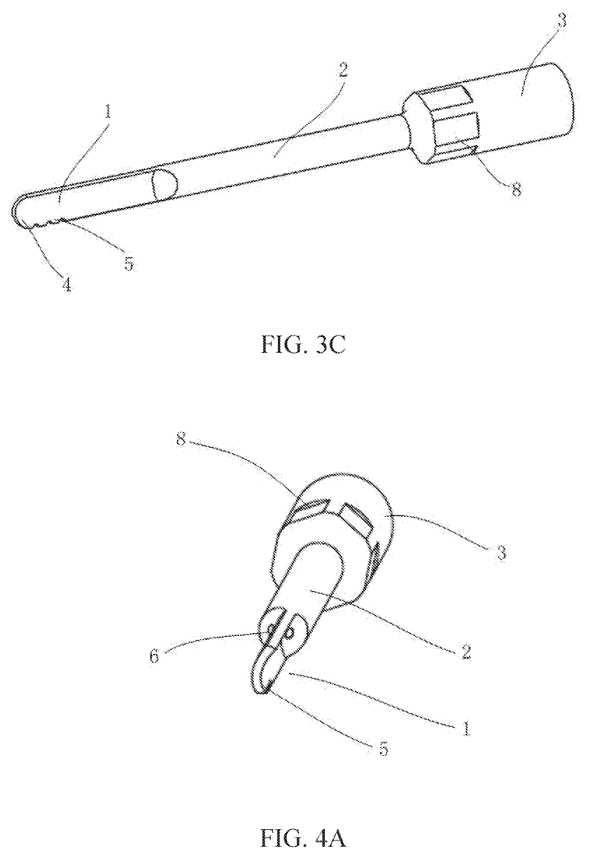

FIG. 3A is a schematic perspective view of a tool bit for an ultrasonic osteotome according to a second embodiment of the present invention, FIG. 3B is a front view of the tool bit for an ultrasonic osteotome according to the second embodiment of the present invention, and FIG. 3C is a schematic perspective view of the tool bit for an ultrasonic osteotome of the second embodiment of the present invention when viewed from another angle. As shown in FIGS. 3A to 3C, in the tool bit for an ultrasonic osteotome according to the second embodiment of the present invention, a curved part 4 is formed at the top of the head portion 1 of the tool bit. The head portion 1 of the tool bit is provided with cutter teeth 5 on a single side thereof. The cutter teeth 5 start from a terminal of the curved part 4. This provides the operator with another way of cutting.

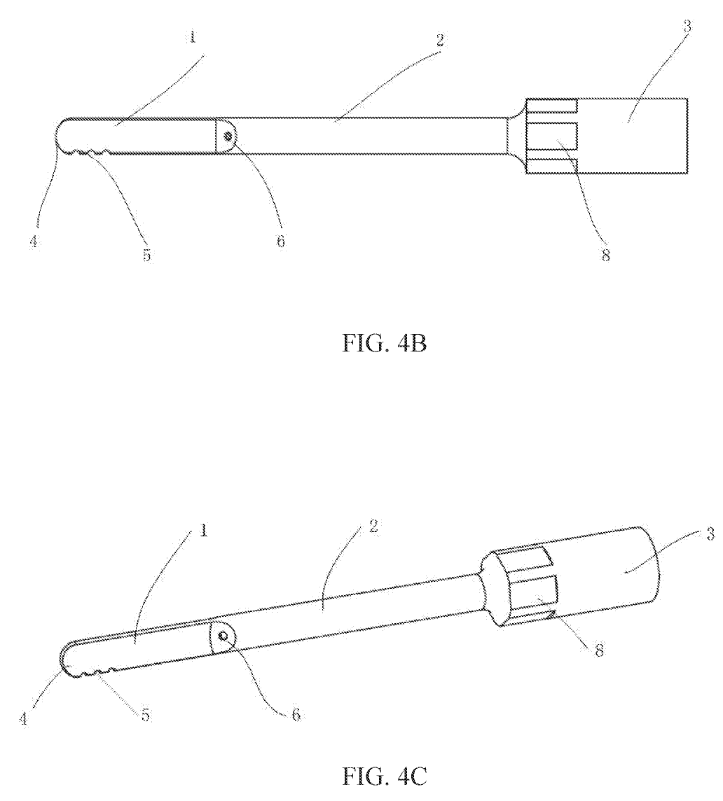

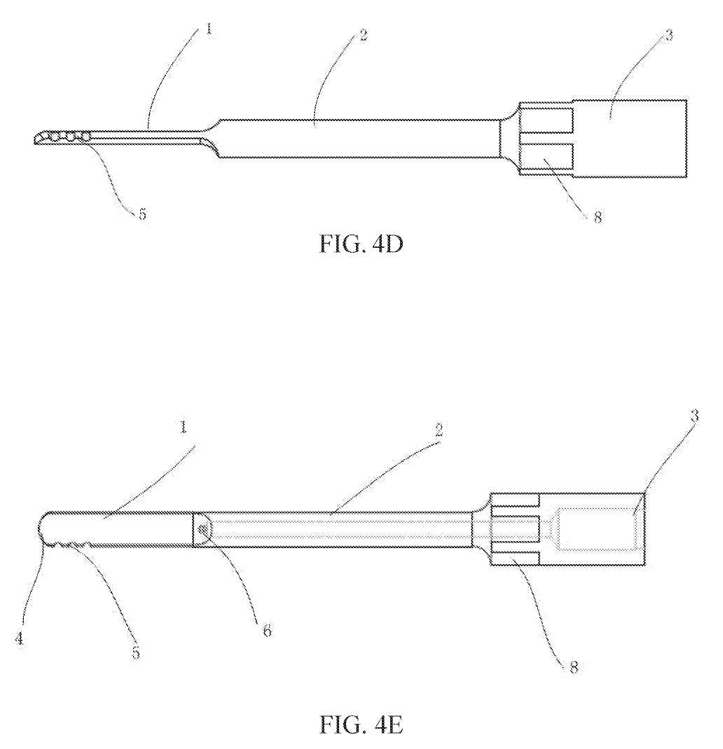

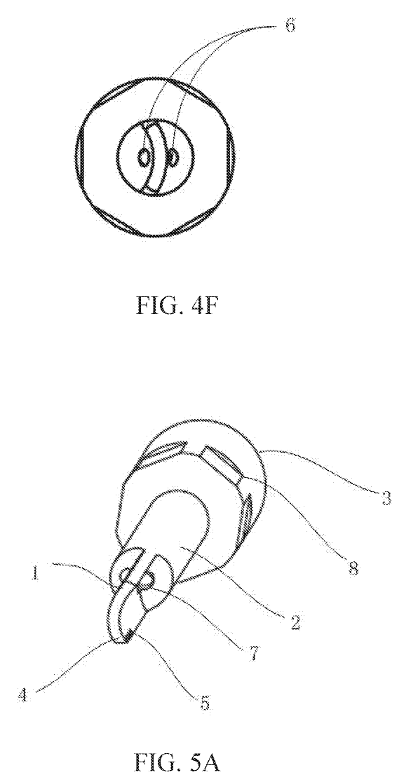

FIG. 4A is a schematic perspective view of a hollow structure of a tool bit for an ultrasonic osteotome according to an embodiment of the present invention. FIG. 4B is a front elevational view showing the tool bit for an ultrasonic osteotome having the hollow structure according to the embodiment of the present invention. FIG. 4C is a schematic perspective view showing the tool bit for an ultrasonic osteotome having the hollow structure according to the embodiment of the present invention viewed from another angle. FIG. 4D is a bottom plan view showing the tool bit for an ultrasonic osteotome having the hollow structure according to the embodiment of the present invention. FIG. 4E is a front elevational view showing an internal hollow structure of the tool bit for an ultrasonic osteotome having the hollow structure according to the embodiment of the present invention. FIG. 4F is a left side view of the tool bit for an ultrasonic osteotome having the hollow structure according to the embodiment of the present invention. As shown in FIGS. 4A to 4F, as a modification of the first embodiment of the present invention, a tool bit for an ultrasonic osteotome of the present invention may be provided with a through hole at a center of the tool bit to form a hollow structure, and a guide hole 6 may be provided at a connection portion between the arbor 2 and the head portion 1 of the tool bit. There may be two guide holes 6 provided at each of an inner surface and an outer surface of the head portion 1 of the tool bit, respectively. The guide holes 6 can ensure to guide water flow to a surgical cutting surface of the head portion 1 of the tool bit, and also have a cooling effect for the tool bit itself. During the operation, the entire head portion of the tool bit and the contacted tissue are completely exposed to the perfusate for cleaning and cooling in real time, ensuring that the excised bone fragments are immediately discharged, and the visual field at the incision is clear and clean so that the tissue to be retained is protected from being damaged. This can further reduce the risk of operation and improve the safety and success rate of surgery. The tool bit of an ultrasonic osteotome with a hollow structure can also reduce the weight of the tool bit to a certain extent, so that the tool bit is smaller and lighter in structure, more labor-saving for gripping, and more convenient for operation.

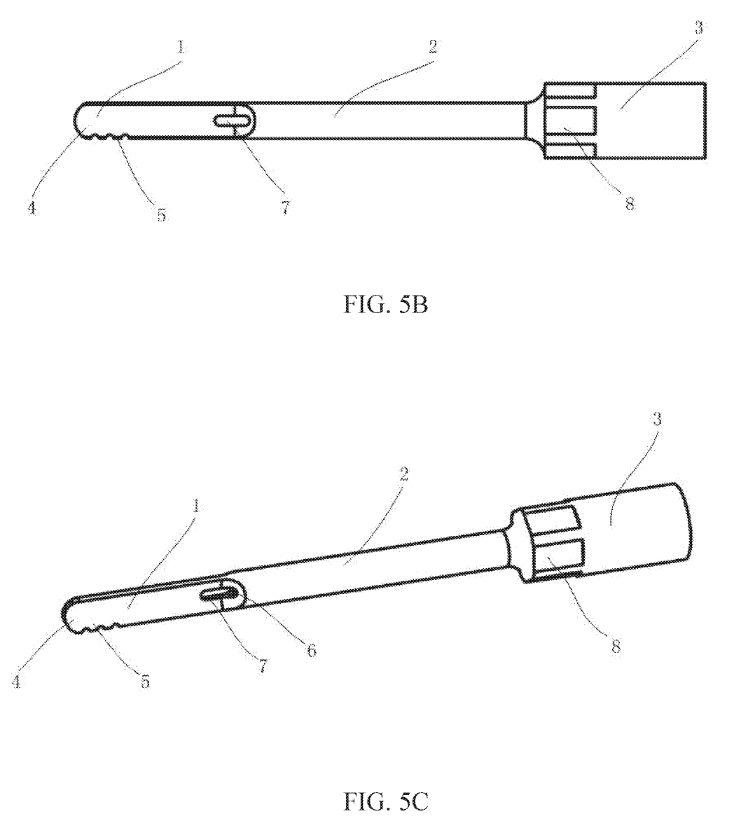

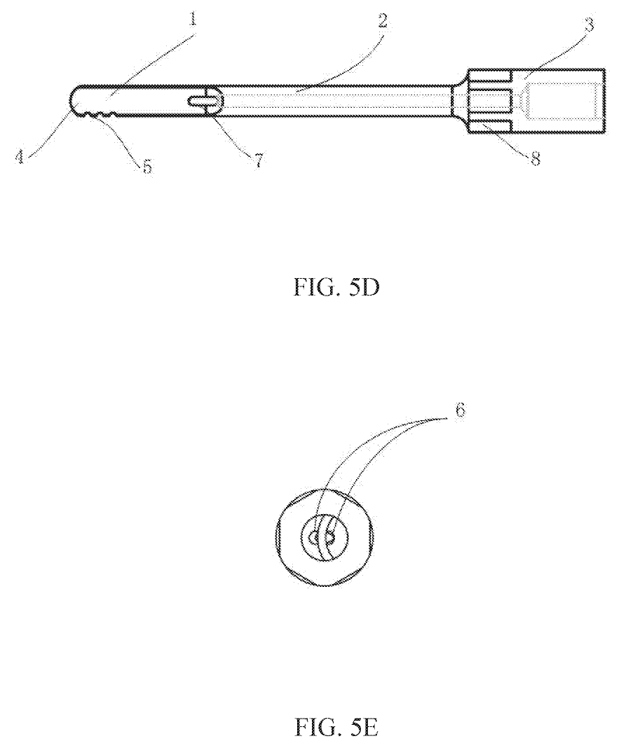

FIG. 5A is a schematic perspective view showing a tool bit for an ultrasonic osteotome having a hollow structure according to another embodiment of the present invention. FIG. 5B is a front elevational view showing the tool bit for an ultrasonic osteotome having the hollow structure according to another embodiment of the present invention. FIG. 5C is a schematic perspective view showing the tool bit for an ultrasonic osteotome having the hollow structure according to another embodiment of the present invention viewed from another angle. FIG. 5D is a front elevational view showing an internal hollow structure of the tool bit for an ultrasonic osteotome having the hollow structure according to another embodiment of the present invention. FIG. 5E is a left side view of the tool bit for an ultrasonic osteotome having the hollow structure according to another embodiment of the present invention. As shown in FIGS. 5A to 5E, the tool bit for an ultrasonic osteotome of the present embodiment has a hollow structure by providing a through hole in a center of the tool bit. A guide hole 6 is provided at a connection portion between the head portion 1 of the tool bit and the arbor 2, and a guide slot 7 is provided in the head portion 1 of the tool bit. The guide slot 7 may be provided only in a part of the head portion 1 of the tool bit, as shown in FIGS. 5B to 5D. The guide slot 7 can also be extended all the way to the front top end of the head portion 1 of the tool bit. The presence of the guide slot 7 can ensure that a greater amount of water flow is guided to a surgical cutting portion at the head portion of the tool bit, providing more adequate cooling of the surgical site of the tool bit. During the operation, the entire head portion of the tool bit and the contacted tissue are completely exposed to a perfusate for cleaning and cooling in real time, ensuring that the bone excised fragments can be discharged more immediately, and the visual field at the incision is clearer and cleaner, and further improving the cleaning and cooling effects in the operation.

In the tool bit for an ultrasonic osteotome of embodiments of the present invention, a connection mechanism may be provided on the bit body 3. Preferably, the connection mechanism is a threaded structure, which may be an external thread or an internal thread. Of course, the connection mechanism is not limited to the threaded structure, any connection mechanism capable of fixing the tool bit for an ultrasonic osteotome of the present invention to a transducer in a detachable manner can be provided on the bit body of the tool bit for an ultrasonic osteotome of the present invention. When the connection thread on the bit body of the tool bit for an ultrasonic osteotome of the present invention is connected to a specific ultrasonic transducer and tightened with a corresponding wrench, and then the ultrasonic transducer is connected to a specific ultrasonic main machine, it is ready to work. The tool bit for an ultrasonic osteotome is suitable for minimally invasive surgery in orthopedic surgery, and can reduce operation time and the risk of breaking off the tool bit by accidence.

Compared with the prior art, the embodiments of the invention have the following advantages: the tool bit for an ultrasonic osteotome of the invention can concentrate all the energy generated by the ultrasonic transducer on the front end portion of the tool bit (the most effective working part), so that the front end portion of the tool bit has the strongest energy output for the strongest working effect. Since a back side portion of a main cutting portion of the head portion of the tool bit, i.e., a back side portion formed with the cutter teeth, adopts a planar or an arcuate structure, it can effectively avoid damage to the spinal cord by scratching when a cutting operation is performed by bypassing the spinal cord, thereby improving the safety of the operation. When the tool bit for an ultrasonic osteotome is used for osteotomy, it can conveniently and quickly cut a hole in the vertebral plate or other bone tissue, thus the tool bit for an ultrasonic osteotome is suitable for minimally invasive surgery in orthopedic surgery, and can reduce operation time.

It should be noted that the above embodiments are only used to describe the concept of the present invention, rather than limiting the present invention. Although detailed descriptions of the invention are made with reference to the above embodiments, it would be appreciated by those skilled in the art that various changes or modifications to the above embodiments may be made or equivalent substitutions to portion of or all features in those embodiments may be made. Such changes, modifications or substitutions will not make the spirit of the relevant solutions depart from the scope of the technical solutions of the embodiments of the present invention.

What is claimed is:

1. A tool bit for an ultrasonic osteotome comprising a head portion of the tool bit, an arbor and a bit body, with one end of the arbor being connected with the head portion of the tool bit, and the other end of the arbor being connected with the bit body, wherein the head portion of the tool bit has a flat-sheet shape, and the head portion of the tool bit is bent about an axis in an axial direction of the tool bit, with an inner surface and an outer surface of the flat-sheet head portion of the tool bit having a same radius of curvature, and the head portion of the tool bit is provided with a curved part.

2. The tool bit for an ultrasonic osteotome of claim 1, wherein the curved part is provided on a single side of the head portion of the tool bit.

3. The tool bit for an ultrasonic osteotome of claim 1, wherein the curved part is provided with cutter teeth.

4. The tool bit for an ultrasonic osteotome of claim 1, wherein the curved part is provided at the top of the head portion of the tool bit.

5. The tool bit for an ultrasonic osteotome of claim 1, wherein the head portion of the tool bit is provided with cutter teeth on a single side thereof.

6. The tool bit for an ultrasonic osteotome of claim 1, wherein the tool bit for an ultrasonic osteotome is of a hollow structure.

7. The tool bit for an ultrasonic osteotome of claim 6, wherein a guide hole is provided at a connection portion between the arbor and the head portion of the tool bit.

8. The tool bit for an ultrasonic osteotome of claim 7, wherein a guide slot is provided in the head portion of the tool bit to communicate with the guide hole.

9. The tool bit for an ultrasonic osteotome of claim 1, wherein the arbor and the bit body are transitioned by a beveled or curved surface.

10. The tool bit for an ultrasonic osteotome of claim 1, wherein the bit body is provided with a connection mechanism at the other end thereof for connection with an ultrasonic transducer, and the bit body is provided with clamping faces on an outer surface thereof for clamping.
